(19) United States
(12) Plant Patent Application Publication
(10) Pub. No.: US 2002/0073468 P1
(43) Pub. Date: Jun. 13, 2002

(54) VARIETY OF GERANIUM PLANT NAMED 'FIWOSCARL'

(76) Inventor: Andrea Michalik, Dresden (DE)

Correspondence Address: Russell D. Orkin, WEBB ZIESENHEIM LOGSDON ORKIN & HANSON, P.C., 700 Koppers Building, 436 Seventh Avenue, Pittsburgh, PA, 15219-1818, US

(21) Appl. No.: 09/733,022

(22) Filed: Dec. 8, 2000

(51) Int. Cl. A01H 5/00

(52) U.S. Cl. PLT/330

(57) ABSTRACT

A new and distinct zonal geranium plant with red colored flowers above medium green foliage. The plant flower is suitable for pot planting or as a bedding plant.

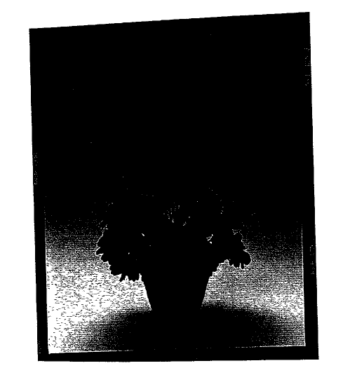

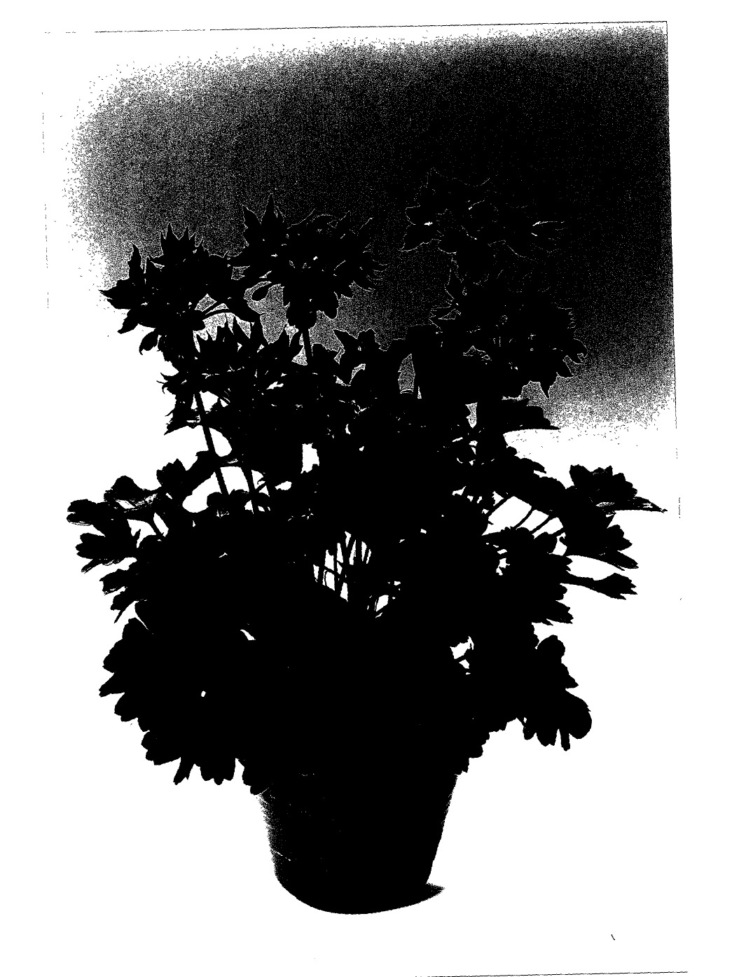

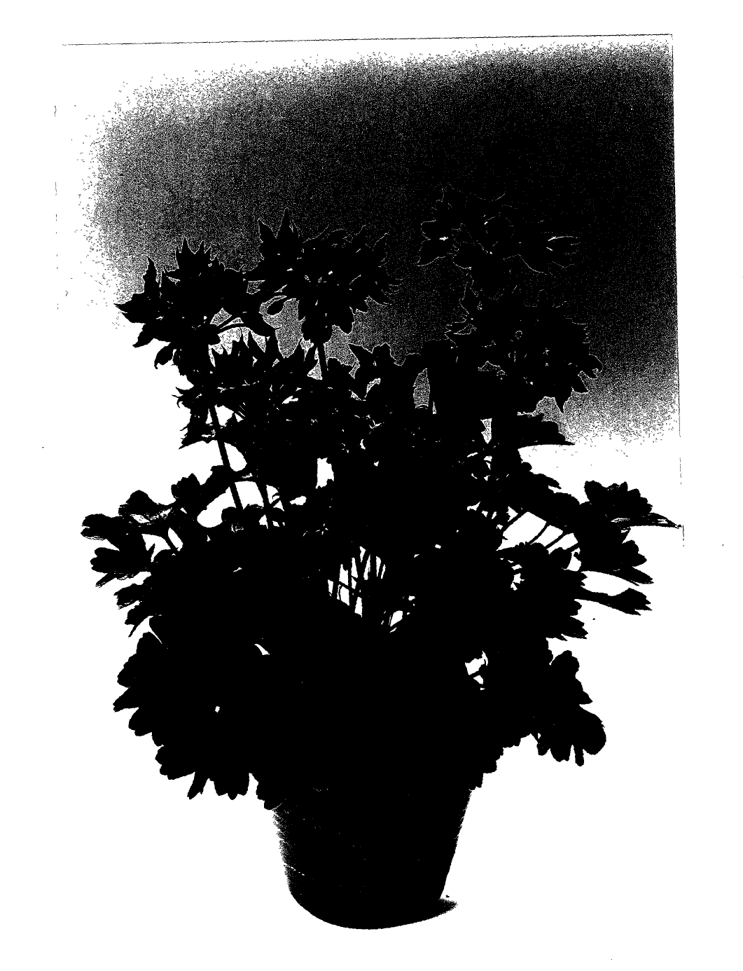

### BACKGROUND OF THE INVENTION

The present invention comprises a new and distinct cultivar of Pelargonium×hortorum known by the varietal name ‘Fiwoscarl’. The new variety was discovered in 1998 in a selective breeding program in Dresden, Germany. The new variety is the result of a combination breeding of 40 stellar-type seedlings having red colored flowers and no leaf zonation. The new variety was first asexually reproduced by cuttings in Dresden, Germany in 1999. The new variety has been trial and field tested and has been found to retain its distinctive characteristics and remain true to type through successive propagations.

### DESCRIPTION OF THE DRAWINGS

The accompanying photographic drawing illustrates the new variety, with the color being as nearly true as is possible with color illustrations of this type.

### DESCRIPTION OF THE PLANT

The following detailed description sets forth the characteristics of the new cultivar. The data which defines these characteristics were collected by asexual reproductions carried out in Dresden, Germany. The color readings were taken outdoors in diffuse light during summer. Color references are primarily to the R.H.S. Colour Chart of The Royal Horticultural Society of London.

Plant:

Classification:

Botanical.—Pelargonium×hortorum.

Commercial.—Zonal geranium.

Form.—Erect. Height from media surface:

Top of foliage.—45 cm.

Top of umbels.—55 cm.

Growth.—Erect, medium branching.

Strength.—Does not require artificial support.

Response time.—3.5 weeks.

Leaves:

Size.—6-8 cm long.

Shape.—Stellar-type.

Margin.—Toothed.

Texture.—Smooth.

Color.—Green Group 139B with prominent zone.

Ribs and veins:

Color.—Darker than leaf surface.

Petioles:

Color.—Light green.

Length.—3-5 cm.

Stem/branches:

Color.—Medium green.

Internode length.—3-5 cm.

The bud:

Shape when just showing color:

Overall.—Semi-spherical.

Individual bud.—Narrow, elliptical.

Color.—Green Group 143B.

Size when just showing color:

Umbel.—6-8 cm across.

Individual bud.—1-1.5 cm long; 0.5 cm wide.

Inflorescence:

Blooming habit.—Star-like; single.

Size of fully open bloom.—2-3 cm.

Borne.—Flowers on umbel; umbel on pedicel; pedicel on peduncle.

Flowers:

Form.—Zygomorph.

Number of petals.—5.

Size:

Upper petals.—0.5-0.7 cm wide; 1.5 cm long.

Lower petals.—0.6-0.8 cm wide; 1.5 cm long.

Petals:

Color:

Upper surface.—Red Group 40A.

Lower surface.—Red Group 40C.

Texture and appearance.—Velvety.

Petaloids.—Absent.

Pedicel:

Length.—2.5-3.0 cm.

Color.—Green-red.

Peduncle:

Length.—10 cm.

Color.—Medium green.

Disease/pest resistance.—No unusual susceptibility to diseases or pests has been noted to date.

Lasting Quality.—Long-lasting; 3-4 weeks per umbel.

Reproductive organs:

Stamens:

Anthers.—3 mm long.

Filaments:

Length.—0.5 cm maximum.

Color.—Bright pink.

Pollen.—Dark yellow.

Pistils:

Number.—Five.

Length.—0.3-0.4 cm.

Stigma.—Red.

Style.—0.8 cm long.

Ovaries:

Color.—Greenish.

Length.—0.4 cm.

Fruit.—Present with 5 seeds; weak fruit production.

### GENERAL CHARACTERISTICS

1. Stellar-shaped foliage.

2. Erect, compact growth.

3. Single, star-like flowers.

4. Harmonic habitus.

5. Long-lasting.

6. Good arrangeability with other colors.

I claim:

1. A new and distinct variety of zonal geranium plant substantially as shown and described.
